(19) United States
(12) Patent Application Publication
(10) Pub. No.: US 2025/0152078 A1
(43) Pub. Date: May 15, 2025

(54) APPARATUS AND METHOD FOR DETECTING VISUAL STIMULUS BASED ON SSVEP

(71) Applicants: HYUNDAI MOTOR COMPANY, Seoul (KR); Kia Corporation, Seoul (KR)

(72) Inventors: Hoseung CHA, Guri-si (KR); Seungkyu NAM, Seoul (KR); Taejun LEE, Suwon-si (KR)

(73) Assignees: HYUNDAI MOTOR COMPANY, Seoul (KR); Kia Corporation, Seoul (KR)

(21) Appl. No.: 18/678,554

(22) Filed: May 30, 2024

(30) Foreign Application Priority Data
Nov. 13, 2023 (KR) 10-2023-0156298

(51) Int. Cl. A61B 5/378; A61B 5/00; A61B 5/375

(52) U.S. Cl. CPC A61B 5/378; A61B 5/375; A61B 5/7267

(57) ABSTRACT

The apparatus for detecting a visual stimulus based on steady-state visual evoked potential (SSVEP) may include a visual stimulus signal receiver configured to receive a visual stimulus signal extracted through an electroencephalogram (EEG) analysis with respect to a user gazing at the visual stimulus of a particular frequency, a visual guide unit configured to dispose a visual guide having a particular form on the visual stimulus, a visual stimulus signal processor configured to classify the visual stimulus based on the received visual stimulus signal and generate a visual feedback, and a visual feedback reflector configured to reflect the generated visual feedback to the visual guide. The visual guide unit may be configured to vary a shape of the visual guide based on the reflected visual feedback.

### CROSS-REFERENCE TO RELATED APPLICATION

This application claims priority to and the benefit of Korean Patent Application No. 10-2023-0156298 filed in the Korean Intellectual Property Office on Nov. 13, 2023, the entire contents of which is incorporated herein by reference.

### TECHNICAL FIELD

The present disclosure relates to an apparatus and method for detecting a visual stimulus based on steady-state visual evoked potential (SSVEP). More particularly, the present disclosure relates to an apparatus and method for detecting a visual stimulus based on SSVEP provided with a visual guide providing a real-time feedback.

### BACKGROUND

A steady-state visual evoked potential (SSVEP) is an electroencephalogram potential generated when gazing at a visual stimulus flickering at a particular frequency, and may be extracted through an electroencephalogram (EEG) analysis measured near the occipital lobe.

Since a particular frequency of the gazed visual stimulus may be detected from the electroencephalogram signal, which visual stimulus was gazed at by the user may be identified through electroencephalogram analysis. Accordingly, it may be utilized in developing various brain-computer interfaces (BCIs).

Despite the development of SSVEP-based BCI algorithms, there is still a problem in which BCI performance is greatly affected by the user fatigue, concentration, and attitude.

In general, in order to detect SSVEP signals with constant performance, a fixed SSVEP stimulation time (fixed time window) is used through prior offline analysis, and the results of the stimulus that the user gazes at may be known after the stimulation ends.

For SSVEP stimulation, there is variability in the way each person looks at the stimulus, and as a result, there is a problem of difficulty in looking at the way they look.

### SUMMARY

The present disclosure attempts to provide an apparatus and method for detecting a visual stimulus based on SSVEP that may include a visual guide configured to provide a real-time feedback according to significance probability.

The present disclosure attempts to provide an apparatus and method for detecting a visual stimulus based on SSVEP capable of intuitively identifying which stimulus the feedback is to by reacting in real time to the stimulus gazed at by the user and provided with a visual guide improving the detecting performance with respect to the visual stimulus by strengthen the user's gazing concentration.

The apparatus for detecting a visual stimulus based on SSVEP may include a visual stimulus signal receiver configured to receive a visual stimulus signal extracted through an electroencephalogram (EEG) analysis with respect to a user gazing at the visual stimulus of a particular frequency, a visual guide unit configured to dispose a visual guide having a particular form on the visual stimulus, a visual stimulus signal processor configured to classify the visual stimulus based on the received visual stimulus signal and generate a visual feedback, and a visual feedback reflector configured to reflect the generated visual feedback to the visual guide. The visual guide unit may be configured to vary a shape of the visual guide based on the reflected visual feedback.

The visual guide may have the same frequency as the particular frequency of the visual stimulus.

The visual guide unit may be configured to vary the shape of the visual guide when the visual stimulus flickers according to the particular frequency.

The visual stimulus may include a checkerboard-based visual stimulus that is inversed according to the particular frequency, and the visual guide may appear on a checkerboard at an inversion time point of the checkerboard, with respect to the checkerboard-based visual stimulus.

The visual stimulus signal processor may include a visual stimulus classifier configured to extract a feature of the visual stimulus signal and classify the visual stimulus based on the extracted feature, and a visual feedback generator configured to generate the visual feedback including feedback information related to classification of the visual stimulus.

The visual stimulus signal processor may be configured to calculate a slope of a regression line for each visual stimulus through an analysis of covariance (ANCOVA) with respect to a plurality of visual stimuli including the visual stimulus, the visual feedback generator may be configured to generate a significance probability with respect to a difference between a first slope appearing largest corresponding to the visual stimulus and a second slope corresponding to another visual stimulus other than the visual stimulus as the visual feedback, and the significance probability may be inversely proportional to a magnitude of the difference of the slope.

The significance probability may have a value varying in a range of 0 or more and 1 or less with respect to the visual stimulus, and the visual feedback generator may be configured to generate a positive first feedback with respect to the classification as the significance probability is closer to 0 and generate a negative second feedback with respect to the classification as the significance probability is closer to 1.

The visual guide unit may be configured to vary the shape of the visual guide to a first shape corresponding to the first feedback and to vary the shape of the visual guide to a second shape corresponding to the second feedback.

The visual feedback reflector may be configured to reflect the visual feedback including the varying significance probability to the visual guide in real time, and the visual guide unit may be configured to, in response to the real-time visual feedback, vary the shape of the visual guide in real time to have a continuous shape dynamically changing between the first shape and the second shape.

When the significance probability satisfies a significance level, the visual stimulus classifier may be configured to classify a stimulus corresponding to a largest canonical correlation analysis (CCA) coefficient among CCA coefficients used in the analysis of covariance as the user's intended visual stimulus.

The visual guide unit may be configured to dispose the visual guide at a center of the visual stimulus.

A visual stimulus detection method may include receiving a visual stimulus signal extracted through an EEG analysis with respect to a user gazing at the visual stimulus of a particular frequency, by an apparatus for detecting visual stimulus based on a steady-state visual evoked potential (SSVEP), disposing a visual guide having a particular form on the visual stimulus, by the apparatus for detecting visual stimulus based on SSVEP, visual stimulus signal processing to classify the visual stimulus based on the received visual stimulus signal and generate a visual feedback, by the apparatus for detecting visual stimulus based on SSVEP, and reflecting the visual feedback to the visual guide in real time, by the apparatus for detecting visual stimulus based on SSVEP. The disposing the visual guide may include varying a shape of the visual guide in real time based on the visual feedback.

The visual guide may have the same frequency as the particular frequency of the visual stimulus.

The disposing the visual guide may further include varying the shape of the visual guide when the visual stimulus flickers according to the particular frequency.

The visual stimulus signal processing may include extracting a feature of the visual stimulus signal and classifying the visual stimulus based on the extracted feature, and generating the visual feedback including feedback information related to classification of the visual stimulus.

The generating the visual feedback may include calculating a slope of a regression line for each visual stimulus through an analysis of covariance with respect to a plurality of visual stimuli including the visual stimulus, and calculating a significance probability with respect to a difference between a first slope appearing largest corresponding to the visual stimulus and a second slope corresponding to another visual stimulus other than the visual stimulus as the visual feedback, where the significance probability may be inversely proportional to a magnitude of the difference of the slope.

The significance probability may have a value varying in a range of 0 or more and 1 or less with respect to the visual stimulus, and the generating the visual feedback may further include generating a positive first feedback with respect to the classification as the significance probability is closer to 0 and generating a negative second feedback with respect to the classification as the significance probability is closer to 1.

The disposing the visual guide may further include varying the shape of the visual guide to a first shape corresponding to the first feedback and varying the shape of the visual guide to a second shape corresponding to the second feedback.

The generating the visual feedback may further include setting all significance probabilities with respect to slope differences between other visual stimuli to 1, excluding the significance probability with respect to the difference between the first slope and the second slope, and a magnitude of the second slope may be subsequently large to magnitude of the first slope.

The disposing the visual guide may further include disposing the visual guide at a center of the visual stimulus.

According to an apparatus and method for detecting a visual stimulus based on SSVEP according to an embodiment, a visual guide that provides real-time feedback related to classification of visual stimuli is included, such that the user may intuitively know which stimulus the feedback is for and the user's SSVEP visual detection performance may be improved by strengthening gaze concentration.

According to an apparatus and method for detecting a visual stimulus based on SSVEP according to an embodiment, usability is high because it may be directly added to visual stimuli suitably designed to induce SSVEP.

According to an apparatus and method for detecting a visual stimulus based on SSVEP according to an embodiment, the visual guide is synchronized to the same frequency as the SSVEP, thereby strengthening the frequency stimulus and improving detection performance.

### BRIEF DESCRIPTION OF THE DRAWINGS

FIG. 1 is an example diagram of a BCI system to which an apparatus for detecting a visual stimulus based on SSVEP according to an embodiment is applied.

FIG. 2 is a block diagram of an apparatus for detecting a visual stimulus based on SSVEP according to an embodiment.

FIG. 3 is a drawing showing a visual stimulus processing process by an apparatus for detecting a visual stimulus based on SSVEP according to an embodiment.

FIG. 4 is a flowchart showing classification of visual stimulus and generation of visual feedback by a visual stimulus signal processor according to an embodiment.

FIG. 5 and FIG. 6 are drawings showing a visual guide according to an embodiment.

FIG. 7 is an example diagram showing an appearance of a visual guide according to the user's gaze according to an embodiment.

FIG. 8 is a flowchart of a visual stimulus detection method based on SSVEP according to an embodiment.

FIG. 9 is a drawing for explaining a computing device according to an embodiment.

### DETAILED DESCRIPTION OF THE EMBODIMENTS

An embodiment of the disclosure will be described more fully hereinafter with reference to the accompanying drawings such that a person skill in the art may easily implement the embodiment. As those skilled in the art would realize, the described embodiments may be modified in various different ways, all without departing from the spirit or scope of the present disclosure. In order to clarify the present disclosure, parts that are not related to the description will be omitted, and the same elements or equivalents are referred to with the same reference numerals throughout the specification.

In addition, unless explicitly described to the contrary, the word “comprise” and variations such as “comprises” or “comprising” will be understood to imply the inclusion of stated elements but not the exclusion of any other elements. Terms including an ordinary number, such as first and second, are used for describing various constituent elements, but the constituent elements are not limited by the terms. The terms are only used to differentiate one component from other components.

In addition, the terms “unit”, “part” or “portion”, “-er”, and “module” in the specification refer to a unit that processes at least one function or operation, which may be implemented by hardware, software, or a combination of hardware and software.

Hereinafter, embodiments of the present disclosure will be described with reference to the drawings.

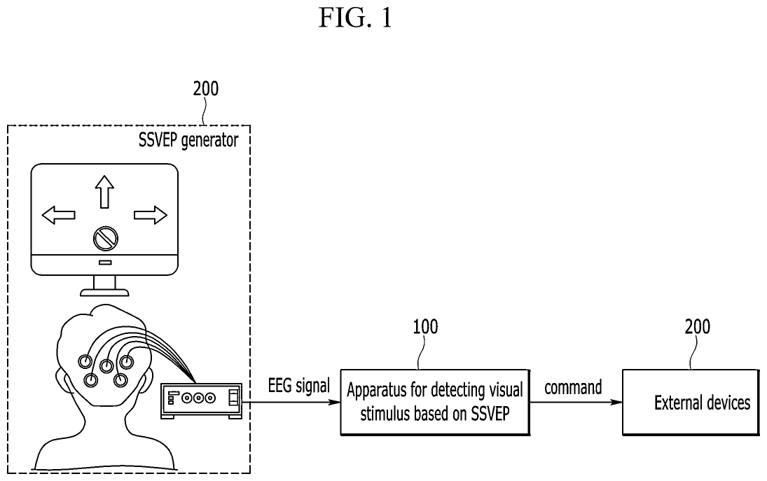

FIG. 1 is an example diagram of a brain-computer interface (BCI) system to which an apparatus for detecting a visual stimulus based on SSVEP according to an embodiment is applied.

Referring to FIG. 1, a BCI system to which an apparatus for detecting a visual stimulus based on SSVEP according to an embodiment is applied may include an apparatus 100 for detecting visual stimulus based on SSVEP (hereinafter referred to as a visual stimulus detection apparatus 100), a steady-state visual evoked potential (SSVEP) generator 200, and external devices 300.

The apparatus 100 for detecting a visual stimulus based on SSVEP is a core gist of the present disclosure, and may determine which visual stimulus a user has gazed at, based on the steady-state visual evoked potential (SSVEP).

Here, the steady-state visual evoked potential (SSVEP) is an electroencephalogram potential generated when gazing at a visual stimulus flickering at a particular frequency, and may be extracted through an electroencephalogram (EEG) analysis measured near the occipital lobe. Since a particular frequency of the gazed visual stimulus may be detected from the electroencephalogram signal, the steady-state visual evoked potential (SSVEP) may be identified through electroencephalogram analysis on which visual stimulus was gazed at by the user.

Therefore, the steady-state visual evoked potential (SSVEP) may be utilized in developing various brain-computer interfaces (BCIs). The steady-state visual evoked potential (SSVEP) may be referred to as a visual stimulus signal.

The apparatus 100 for detecting a visual stimulus based on SSVEP according to an embodiment may include a visual guide reacting in real time to the visual stimulus gazed at by the user to provide real-time feedback to the user such that the visual stimulus gazed at by the user may be detected more rapidly and accurately detect.

The SSVEP generator 200 may provide the user with visual stimulation corresponding to the control command with respect to the external device 300, and may induce the user to produce an electroencephalogram (EEG) signal including an electroencephalogram corresponding to the visual stimulus.

For example, when the user gazes at an arrow in the forward direction, an electroencephalogram corresponding to the forward direction is included in the EEG signal of the user. Therefore, the apparatus 100 for detecting a visual stimulus based on SSVEP may detect the electroencephalogram corresponding to the arrow in the forward direction from the EEG signal of the user. That is, the SSVEP generator 200 may transfer the EEG signal to the apparatus 100 for detecting a visual stimulus based on SSVEP.

The external device 300 may be connected to the apparatus 100 for detecting a visual stimulus based on SSVEP through a network. The external device 300 may communicate with the apparatus 100 for detecting a visual stimulus based on SSVEP, and may be controlled according to the command received from the apparatus 100 for detecting a visual stimulus based on SSVEP.

The external device 300 may include a personal mobility, and may include a wheelchair, an exoskeleton, or the like.

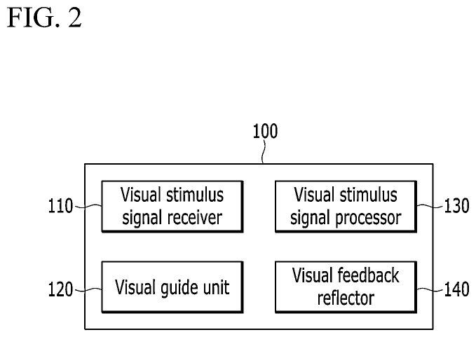

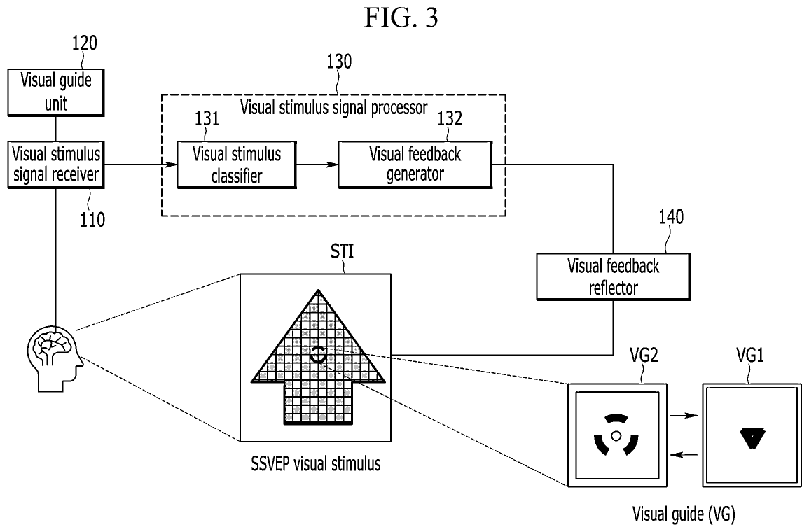

FIG. 2 is a block diagram of an apparatus for detecting a visual stimulus based on SSVEP according to an embodiment. FIG. 3 is a drawing showing a visual stimulus processing process by an apparatus for detecting a visual stimulus based on SSVEP according to an embodiment.

Referring to FIG. 2 and FIG. 3, the apparatus 100 for detecting a visual stimulus based on SSVEP may include a visual stimulus signal receiver 110, the visual guide unit 120, a visual stimulus signal processor 130 and a visual feedback reflector 140.

The visual stimulus signal receiver 110 may receive the visual stimulus signal (SSVEP) extracted through EEG analysis with respect to the user gazing at a visual stimulus STI of the particular frequency.

The visual stimulus STI may be an image that flickers according to the particular frequency. The visual stimulus STI may be a checkerboard image repeatedly inversed according to the particular frequency.

For example, the visual stimulus signal receiver 110 may receive the visual stimulus signal extracted from the electroencephalogram signal of the user gazing at the visual stimulus STI including an image flickering 10 times in 1 second at a frequency of 10 Hz.

The visual guide unit 120 may dispose the visual guide VG having a particular form on the visual stimulus STI. The visual guide unit 120 may dispose the visual guide VG at a center of the visual stimulus STI.

The visual guide VG may improve gaze concentration of the user. The shape, pattern, and color forming the form of the visual guide VG are not limited to FIG. 3, and may be freely set.

In an embodiment, the visual guide VG may have the same frequency as the particular frequency that the visual stimulus STI gazed at by the user has. That is, the visual guide VG may be synchronized with the frequency of the visual stimulus STI gazed at by the user, and strengthen the frequency stimulus transferred to the user. For example, the visual guide VG may flicker at the same frequency as the visual stimulus STI.

The visual stimulus signal processor 130 may classify the visual stimulus STI based on the received visual stimulus signal and may generate a visual feedback. The visual stimulus signal processor 130 may classify the visual stimulus gazed at by the user from other visual stimuli based on the visual stimulus signal. Referring to FIG. 3, the visual stimulus signal processor 130 may include a visual stimulus classifier 131 and the visual feedback generator 132.

The visual stimulus classifier 131 may extract the feature of the visual stimulus signal received from the visual stimulus signal receiver 110, and may classify the visual stimulus STI based on the extracted feature.

The visual stimulus classifier 131 may extract features of the visual stimulus signal. Feature extraction is the process of extracting important information from electroencephalogram signals. The most important feature in the visual stimulus signal (SSVEP) may the frequency of the electroencephalogram.

For example, the visual stimulus classifier 131 may extract the frequency component from the electroencephalogram signal by using Fourier transform or wavelet transform, or the like. The visual stimulus classifier 131 may identify to which visual stimulus the user has reacted to, based on magnitude of power of each frequency component.

The visual stimulus classifier 131 may classify the visual stimulus STI based on the extracted feature. Classification is the process of allocating electroencephalogram signals to a specific class or category using the extracted features.

That is, when the user concentrates on a particular visual stimulus, an electroencephalogram response corresponding to the frequency of the visual stimulus may be generated, and the visual stimulus classifier 131 may determine, through the classification, which stimulus the electroencephalogram signal was in response to. In an embodiment, the classification algorithm may be based on machine-learning techniques. For example, the visual stimulus classifier 131 may classify the visual stimulus by using support vector machine (SVM), K-nearest neighbor (K-NN), linear determination analysis (LDA), or the like.

In an embodiment, the visual stimulus classifier 131 may periodically calculate a correlation coefficient (or CCA coefficient) between a plurality of reference signals having different stimulus frequencies and a multi-channel EEG signal of the user for a reference time, perform an analysis of covariance (ANCOVA) on a plurality of correlation coefficients for each calculated stimulus (stimulus frequency) to calculate a slope of a regression line for each stimulus, and classify the user's intended stimulus through a multiple comparison between the slopes of the regression lines for the respective the calculated stimuli.

Here, multiple comparison means the process of calculating a significance probability P-value with respect to a difference from each remaining slope, respectively, with reference to the largest slope among the calculate slopes of the regression lines for respective stimuli.

For example, when the slope is large in the order of a first visual stimulus, a second visual stimulus, a third visual stimulus, the visual stimulus classifier 131 may be configured to calculate a first significance probability between a slope of the first visual stimulus and a slope of the second visual stimulus, and calculate a second significance probability between the slope of the first visual stimulus and a slope of the third visual stimulus.

When respective significance probabilities calculated through the multiple comparison all satisfy a significance level (significance probability<significance level), the visual stimulus classifier 131 may determine that there exists a statistically significant difference, and may detect a stimulus corresponding to the largest correlation coefficient among the plurality of correlation coefficients as the user's intended stimulus.

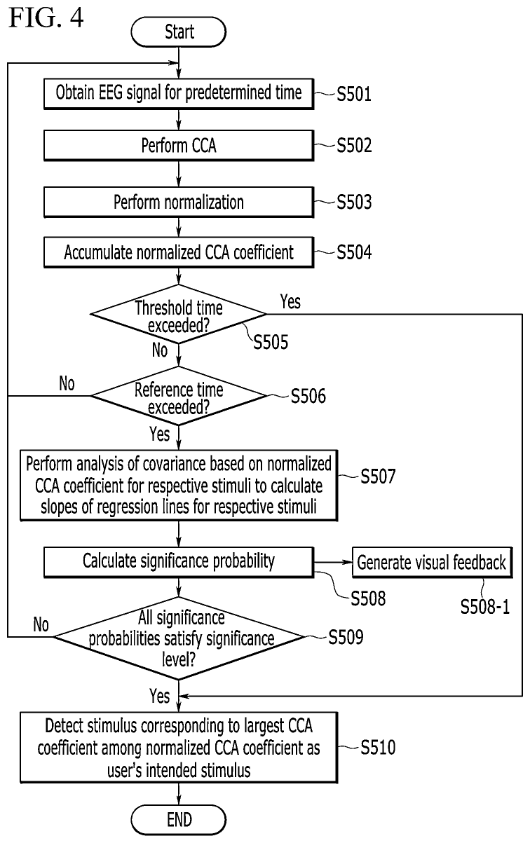

Here, the significance level may be set to one of 0.05, 0.01, and 0.001, for example. Detailed description will be provided with reference to FIG. 4.

The visual feedback generator 132 may generate the visual feedback including feedback information related to classification of visual stimuli.

The visual feedback generator 132 may generate a feedback with respect to the visual stimulus being gazed at by the user as the visual feedback. When the user is gazing at a particular visual stimulus, the visual feedback may include statistics-based information extracted with respect to the visual stimulus. In addition, the visual feedback may include the gaze concentration of the user with respect to particular visual stimuli.

The visual feedback generator 132 may generate the visual feedback by comparing the slopes between the regression lines calculated for each visual stimulus through the analysis of covariance (ANCOVA) with respect to a plurality of visual stimuli.

The visual feedback generator 132 may generate the significance probability P-value with respect to a difference between a first slope appearing largest corresponding to the visual stimulus STI gazed at by the user and a second slope corresponding to another visual stimulus that is not gazed at by the user as the visual feedback.

Unlike the visual stimulus classifier 131, the visual feedback generator 132 requires only the significance probability P-value, and thus slopes for all visual stimuli need not be compared. That is, the visual feedback generator 132 may generate the visual feedback based on the significance probability P-value with respect to the slope difference between the first visual stimulus, which is the visual stimulus STI having the largest first slope and the second visual stimuli having the subsequently largest second slope.

That is, the visual feedback may reflect in real time the significance probability P-value with respect to the difference between the first slope of the first visual stimulus and the second slope of the second visual stimulus.

The significance probability P-value may have a value varying in the range of 0 or more and 1 or less. The significance probability P-value may be inversely proportional to a magnitude of the difference of the slope. For example, as the difference between the first slope and the second slope is greater, the significance probability P-value is closer to 0. As the difference between the first slope and the second slope is smaller, the significance probability P-value is closer to 1.

In an embodiment, the visual feedback generator 132 may preset the significance probability P-value as 1, for visual stimuli other than the first visual stimulus having the largest slope.

The visual feedback generator 132 may generate a first feedback as the significance probability P-value is closer to 0, and may generate a second feedback as the significance probability P-value is closer to 1. The first feedback may be a positive feedback with respect to the classification of the visual stimulus STI, and the second feedback may be a negative feedback with respect to the classification of the visual stimulus STI.

That is, the visual feedback may appear as the first feedback with respect to the visual stimulus STI as the significance probability P-value is closer to 0, and may appear as the second feedback with respect to the visual stimulus STI as the significance probability P-value is closer to 1.

The first feedback may represent that the gaze concentration with respect to the visual stimulus STI gazed at by the user is a high level, and the second feedback may represent that the gaze concentration of the user with respect to the visual stimulus STI is a low level. The visual feedback generator 132 may generate the visual feedback representing the gaze concentration of the user.

The visual feedback reflector 140 may reflect the generated visual feedback to the visual guide VG. That is, the visual feedback reflector 140 may reflect the real-time change of the significance probability P-value between the first visual stimulus and the second visual stimulus to the visual guide VG.

For example, the visual feedback reflector 140 may directly vary a shape of the visual guide VG based on the visual feedback. Alternatively, the visual feedback reflector 140 may transfer the visual feedback to the visual guide unit 120, and may reflect it to the visual guide VG through the visual guide unit 120 in various methods.

In an embodiment, the visual feedback reflector 140 may reflect the gaze concentration with respect to the visual stimulus STI of the user appearing through the visual feedback to the visual guide VG.

The visual guide unit 120 may vary the shape of the visual guide VG based on the visual feedback reflected from the visual feedback reflector 140. That is, the visual guide VG may have a shape that dynamically changes based on the visual feedback.

For example, the visual guide unit 120 may reflect the real-time change of the significance probability P-value between the first visual stimulus and the second visual stimulus, and may vary the shape of the visual guide VG disposed on the visual stimulus STI in real time.

For example, the visual guide unit 120 may vary the shape of the visual guide VG to a first shape VG1 corresponding to the first feedback and may vary the shape of the visual guide VG to a second shape VG2 corresponding to the second feedback. The first shape VG1 and the second shape VG2 are in different shapes but are not particularly limited to FIG. 3.

In response to the real-time visual feedback, the visual guide unit 120 may vary the shape of the visual guide VG in real time, to have a continuous shape dynamically changing between the first shape VG1 and the second shape VG2. In FIG. 3, the visual guide VG may the graphical element of the particular shape.

For example, the visual guide VG may be configured as three parts. Three parts of the visual guide VG may be separated from each other in the second shape VG2. The three parts of the visual guide VG may be coupled from each other in the first shape VG1.

The three parts of the visual guide VG may move away from or become closer to each other, reflecting the real-time visual feedback. As the significance probability P-value is closer to 0, the three parts gets closer to each other, and as the significance probability P-value is closer to 1, the three parts may become further apart from each other.

In an embodiment, the visual guide unit 120 may represent the gaze concentration of the user reflected to the visual feedback through the visual guide VG. That is, the longer the shape of the visual guide VG that dynamically varies by reflecting the significance probability P-value is maintained as the first shape VG1 (i.e., the closer to 0, the significance probability is maintained), the higher the gazing concentration of the user may appear. At this time, there is no need to set the significance level. Therefore, the shape of the visual guide VG may be varied according to the significance probability, regardless of the significance level.

In another embodiment, the visual guide unit 120 may communicate with the visual feedback reflector 140 in order to improve the gaze concentration of the user, and may receive the change of the significance probability P-value due to the shape of the visual guide VG. The visual guide unit 120 may dynamically vary the shape of the visual guide VG in a direction in which the significance probability P-value is maintained close to 0.

FIG. 4 is a flowchart showing classification of visual stimulus and generation of the visual feedback by the visual stimulus signal processor according to an embodiment. FIG. 4 shows the process of calculating the significance probability P-value used for the visual feedback.

First, at step S501, the visual stimulus signal receiver 110 may obtain the EEG signal for a preset time (e.g., 0.175 sec.). At this time, preset time may refer to the time required to obtain the EEG signal that is minimally required to perform a canonical correlation analysis (CCA).

Thereafter, at step S502, the visual stimulus signal processor 130 may perform the CCA on the plurality of visual stimulus signals of visual stimuli having different frequencies and the multi-channel EEG signal of the user to calculate the CCA coefficient for each visual stimulus.

Thereafter, at step S503, the visual stimulus signal processor 130 may perform softmax on the CCA coefficients for each visual stimulus to normalize them. In an embodiment, the visual stimulus signal processor 130 may perform normalization by using L1-norm instead of softmax, in order to generate the visual feedback.

Thereafter, at step S504, the visual stimulus signal processor 130 may accumulate the normalized CCA coefficient for each visual stimulus. For example, the visual stimulus signal processor 130 may accumulate data for 0.5 seconds.

Thereafter, at step S505, the visual stimulus signal processor 130 may determine whether a threshold time (e.g., 4 sec.) has been lapsed after starting the logic.

As a result of the determining at the step S505, when the threshold time is not exceeded, the process may proceed to step S506, and when the threshold time is exceeded, the process may proceed to step S510.

Thereafter, at step S506, the visual stimulus signal processor 130 may determine whether the reference time (minimum window length or detection time) has been lapsed after starting the logic. For example, the reference time may be 0.5 seconds.

When the reference time has not been exceeded, the process may proceed to step S501. When the reference time has been exceeded, at step S507, the visual stimulus signal processor 130 may perform the analysis of covariance based on the normalized CCA coefficient for each stimulus, to calculate the slope of the regression line for each visual stimulus with respect to the plurality of visual stimuli.

Thereafter, at step S508, the visual stimulus signal processor 130 may calculate the significance probability P-value.

In an embodiment, in the perspective of generating the visual feedback, the visual stimulus signal processor 130 may only calculate the significance probability P-value with respect to the slope difference between the largest slope and the subsequently largest slope. This is because the visual feedback may only require the significance probability P-value with respect to the actual visual stimulus STI (refer to FIG. 3). The visual stimulus signal processor 130 may set the significance probability P-value with respect to other visual stimuli as 1.

At step S508-1, the visual stimulus signal processor 130 may generate the visual feedback with the calculated significance probability P-value.

The time point at which the visual guide VG reacts after the visual feedback is generated is from the reference time (minimum window length), which is the time point of beginning calculating the significance probability P-value. For example, the visual feedback may be generated based on the significance probability P-value value when 0.5 seconds after receiving the visual stimulus signal is lapsed.

In an embodiment, the visual stimulus signal processor 130 may perform the multiple comparison to classify visual stimuli to calculate a plurality of significance probabilities P-value. That is, the visual stimulus signal processor 130 may calculate the significance probabilities P-value with respect to a difference from each remaining slope, respectively, with reference to the largest slope among the calculated slopes of the regression lines for respective visual stimuli. The visual stimulus signal processor 130 may determine whether the significance level is satisfied afterwards with the significance probabilities P-value calculated here.

At step S509, the visual stimulus signal processor 130 may determine whether the significance probabilities P-value all satisfy the significance level.

The determination result S509, when any of the significance probabilities P-value does not satisfy the significance level, the process proceeds to the step S501, and when all of the significance probabilities P-value satisfy the significance level, the visual stimulus signal processor 130 may detect the visual stimulus corresponding to the largest CCA coefficient among the normalized CCA coefficients as the user's intended visual stimulus, at step S510.

In an embodiment, the visual stimulus signal processor 130 derives the significance probability P-value based on the slope of the regression line change for each visual stimulus, and accordingly, an initial slope setting of from 0 second to the reference time (e.g., 0.5 sec.) of obtaining the regression line may be offset as the slope with respect to the number of entire visual stimuli.

In the above, although the CCA method that does not require training is taken as an example, a subject-independent filter bank canonical correlation analysis (FBCCA) method, a subject-dependent task-related component analysis (TRCA) method, or the like may also be used.

At this time, as another embodiment, when the FBCCA method is used, the step S502 may be replaced with “At step S502, the visual stimulus signal processor 130 may perform FBCCA on the plurality of reference signals having different stimulus frequencies and the multi-channel EEG signal of the user to calculate the FBCCA coefficient for each stimulus.”

In addition, as a still another embodiment of the present disclosure, when the TRCA method is used, step S502 may be replaced with “At step S502, the visual stimulus signal processor 130 may perform TRCA on the plurality of reference signals having different stimulus frequencies the multi-channel EEG signal of the user to calculate the TRCA coefficient for each stimulus.”

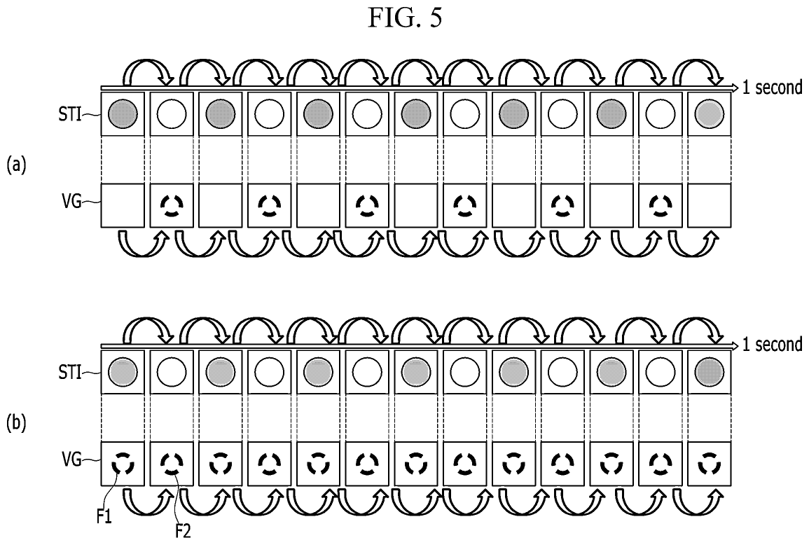

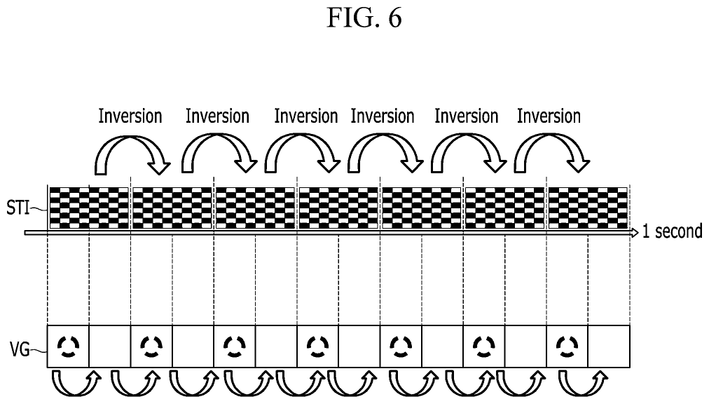

FIG. 5 and FIG. 6 are drawings showing the visual guide according to an embodiment.

FIG. 5 shows flickering of the visual stimulus STI and change of the visual guide VG having a frequency of 6 Hz. (a) in FIG. 5 shows the visual guide VG that flickers at the same frequency whenever the visual stimulus STI having the frequency of 6 Hz flickers.

(b) in FIG. 5 shows the visual guide VG that changes its shape whenever the visual stimulus STI flickers in the visual stimulus STI flickering at the frequency of 6 Hz.

In the embodiment of (a) in FIG. 5, the visual guide VG appears when the visual stimulus STI is while and disappears when it is black. That is, the visual guide VG may flicker at the same frequency as the visual stimulus STI.

In the embodiment according to (b) in FIG. 5, the visual guide unit 120 may vary shape of the visual guide VG whenever the visual stimulus STI flickers according to the particular frequency.

The visual guide unit 120 may have a first shape F1 when the visual stimulus STI is black, and may have a second shape F2 when the visual stimulus STI is white. The first shape F1 and the second shape F2 of FIG. 5 are a mere example, and the shape change of the visual guide VG is not limited thereto. That is, the visual guide VG may be continuously exposed while changing its shape and/or pattern.

FIG. 6 shows a checkerboard that is inversed at 1-second intervals and the change of the visual guide VG corresponding thereto, with respect to the visual stimulus STI having the frequency of 6 Hz.

In an embodiment, the visual stimulus STI may include a checkerboard-based visual stimulus that is inversed according to the particular frequency, and the visual guide VG may appear on the checkerboard with respect to the checkerboard-based visual stimulus at an inversion time point of the checkerboard. That is, the checkerboard-based visual stimulus STI is merely inversed but does not disappear, and the visual guide VG may repeat disappearing after appearing at the inversion time point of the checkerboard, and appearing again at the next inversion time point.

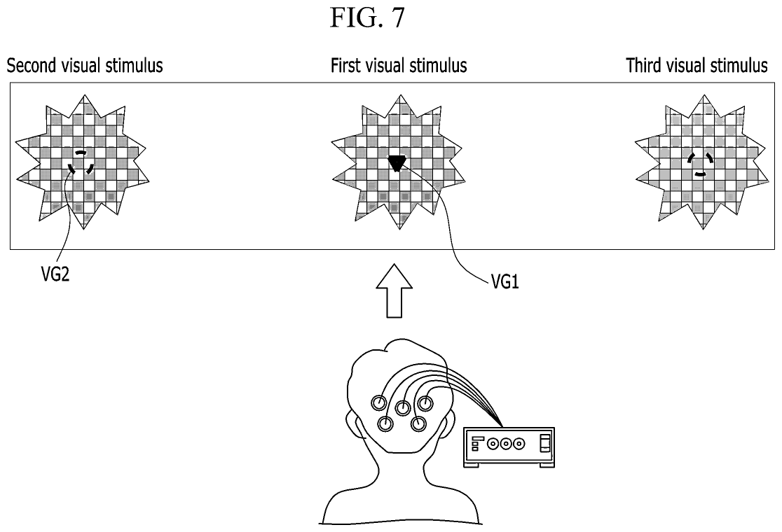

FIG. 7 is an example diagram showing an appearance of the visual guide according to the user's gaze according to an embodiment.

In FIG. 7, the visual guide disposed on the first visual stimulus gazed at by the user may have a first form VG1. However, it may be seen that the visual guide of the second visual stimulus and the third visual stimulus that are not gazed at by the user may have a second form VG2. If the user gases at the second visual stimulus afterwards, the visual guide of the first visual stimulus may change to the second form VG2, and the visual guide of the second visual stimulus may change to the first form VG1.

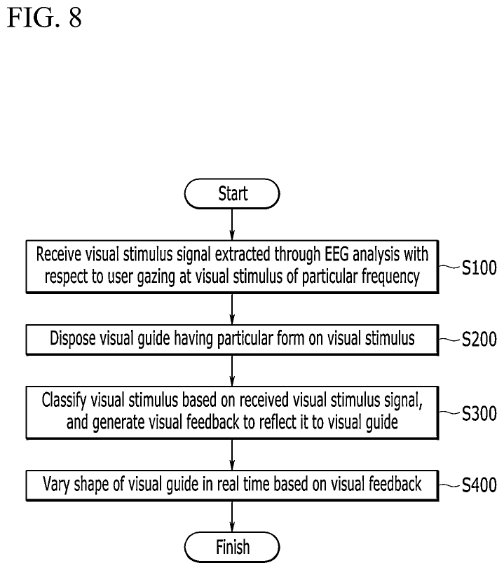

FIG. 8 is a flowchart of a visual stimulus detection method based on SSVEP according to an embodiment. The visual stimulus detection method based on the SSVEP may be performed through the apparatus 100 for detecting visual stimulus based on SSVEP (refer to FIG. 1).

In FIG. 8, at step S100, the apparatus 100 for detecting visual stimulus based on SSVEP (hereinafter referred to as a visual stimulus detection apparatus) may receive the visual stimulus signal extracted through EEG analysis with respect to the user gazing at the visual stimulus of the particular frequency.

At step S200, the visual stimulus detection apparatus 100 may dispose a visual guide having a particular form on the visual stimulus. The visual guide and the visual stimulus may have the same frequency. The visual guide may flicker at the same frequency as the visual stimulus. The shape and pattern of the visual guide may vary in various ways whenever the visual stimulus flickers. The visual guide may be disposed in a center of the visual stimulus.

At step S300, the visual stimulus detection apparatus 100 may classify visual stimulus based on the received visual stimulus signal, and generate the visual feedback to reflect it to the visual guide in real time. In an embodiment, the visual stimulus detection apparatus 100 may extract the features of the visual stimulus signal and classify the visual stimulus based on the extracted feature. The feature may be calculated as the slope of the regression line for each visual stimulus through the analysis of covariance (ANCOVA).

The visual feedback may be generated based on the significance probability P-value with respect to the difference of the slope of the regression line for each visual stimulus. The significance probability P-value may have a value between 0 to 1, and may be inversely proportional to the magnitude of the slope difference. That is, the greater the slope difference between the visual stimulus STI (refer to FIG. 3) having the regression line of the largest slope and other visual stimuli (having the regression line of the subsequently largest slope), the closer to 0 the significance probability may be. The significance probability with respect to the slope difference between other visual stimuli may be set to 1.

The slope difference may represent the degree of classification with respect to the visual stimulus STI. That is, the closer to 0 the significance probability is, the greater the degree of classification from other visual stimuli of the visual stimulus STI is, and the visual stimulus detection apparatus 100 may detect the visual stimulus STI. Classification of each stimulus and generation of the visual feedback may be referred to the description made with reference to FIG. 4.

At step S400, the visual stimulus detection apparatus 100 may vary the shape of the visual guide in real time based on the visual feedback. The shape of the visual guide VG (refer to FIG. 3) may be varied to the first shape by reflecting the generated first feedback as the significance probability is closer to 0, and may be varied to the second shape by reflecting the generated second feedback as the significance probability is closer to 1. The visual guide VG may have a continuous shape that dynamically changes between the first shape and the second shape.

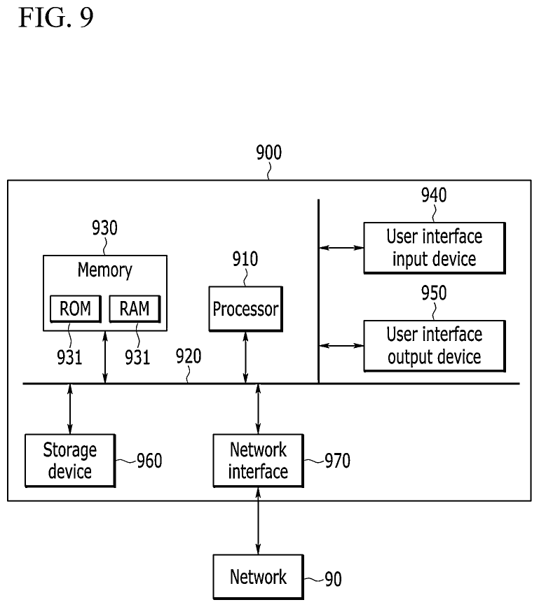

FIG. 9 is drawing for explaining a computing device according to an embodiment.

Referring to FIG. 9, an apparatus and method for detecting a visual stimulus based on SSVEP according to an embodiment may be implemented by using a computing device 900.

The computing device 900 may include at least one of a processor 910, a memory 930, the user interface input device 940, the user interface output device 950 and a storage device 960 that communicate through a bus 920. The computing device 900 may also include a network interface 970 electrically connected to a network 90. The network interface 970 may transmit or receive signals with other entities through the network 90.

The processor 910 may be implemented in various types such as a micro controller unit (MCU), an application processor (AP), a central processing unit (CPU), a graphic processing unit (GPU), a neural processing unit (NPU), and the like, and may be any type of semiconductor device capable of executing instructions stored in the memory 930 or the storage device 960. The processor 910 may be configured to implement the functions and methods described above with respect to FIG. 1 to FIG. 8. For example, various elements described with reference to FIG. 1 to FIG. 8 may be implemented, as a whole or individually, by the processor 910 such that the processor 910 may be configured to implement the functions and methods described above.

The memory 930 and the storage device 960 may include various types of volatile or non-volatile storage media. For example, the memory may include read-only memory (ROM) 931 and a random-access memory (RAM) 932. In this embodiment, the memory 930 may be located inside or outside the processor 910, and the memory 930 may be connected to the processor 910 through various known means.

In some embodiments, at least some configurations or functions of the apparatus and method for detecting a visual stimulus based on SSVEP according to an embodiment may be implemented as a program or software executable by the computing device 900, and program or software may be stored in a computer-readable medium.

In some embodiments, at least some configurations or functions of the apparatus and method for detecting a visual stimulus based on SSVEP according to an embodiment may be implemented by using hardware or circuitry of the computing device 900, or may also be implemented as separate hardware or circuitry that may be electrically connected to the computing device 900.

While this disclosure has been described in connection with what is presently considered to be practical embodiments, it is to be understood that the disclosure is not limited to the disclosed embodiments, but, on the contrary, is intended to cover various modifications and equivalent arrangements included within the spirit and scope of the appended claims.

What is claimed is:

1. The apparatus for detecting a visual stimulus based on steady-state visual evoked potential (SSVEP), comprising:
a visual stimulus signal receiver configured to receive a visual stimulus signal extracted through an electroencephalogram (EEG) analysis with respect to a user gazing at the visual stimulus of a particular frequency;
a visual guide unit configured to dispose a visual guide having a particular form on the visual stimulus;
a visual stimulus signal processor configured to classify the visual stimulus based on the received visual stimulus signal and generate a visual feedback; and
a visual feedback reflector configured to reflect the generated visual feedback to the visual guide,
wherein the visual guide unit is configured to vary a shape of the visual guide based on the reflected visual feedback.

2. The apparatus of claim 1, wherein the visual guide has the same frequency as the particular frequency of the visual stimulus.

3. The apparatus of claim 2, wherein the visual guide unit is configured to vary the shape of the visual guide when the visual stimulus flickers according to the particular frequency.

4. The apparatus of claim 1, wherein:
the visual stimulus includes a checkerboard-based visual stimulus that is inversed according to the particular frequency; and
the visual guide appears on a checkerboard at an inversion time point of the checkerboard, with respect to the checkerboard-based visual stimulus.

5. The apparatus of claim 1, wherein the visual stimulus signal processor comprises:
a visual stimulus classifier configured to extract a feature of the visual stimulus signal and classify the visual stimulus based on the extracted feature; and
a visual feedback generator configured to generate the visual feedback including feedback information related to classification of the visual stimulus.

6. The apparatus of claim 5, wherein:
the visual stimulus signal processor is configured to calculate a slope of a regression line for each visual stimulus through an analysis of covariance (ANCOVA) with respect to a plurality of visual stimuli including the visual stimulus;
the visual feedback generator is configured to generate a significance probability with respect to a difference between a first slope appearing largest corresponding to the visual stimulus and a second slope corresponding to another visual stimulus other than the visual stimulus as the visual feedback; and
the significance probability is inversely proportional to a magnitude of the difference of the slope.

7. The apparatus of claim 6, wherein:
the significance probability has a value varying in a range of 0 or more and 1 or less with respect to the visual stimulus; and
the visual feedback generator is configured to generate a positive first feedback with respect to the classification as the significance probability is closer to 0 and generate a negative second feedback with respect to the classification as the significance probability is closer to 1.

8. The apparatus of claim 7, wherein the visual guide unit is configured to vary the shape of the visual guide to a first shape corresponding to the first feedback and to vary the shape of the visual guide to a second shape corresponding to the second feedback.

9. The apparatus of claim 8, wherein:
the visual feedback reflector is configured to reflect the visual feedback including the varying significance probability to the visual guide in real time; and
the visual guide unit is configured to, in response to the real-time visual feedback, vary the shape of the visual guide in real time to have a continuous shape dynamically changing between the first shape and the second shape.

10. The apparatus of claim 6, wherein, when the significance probability satisfies a significance level, the visual stimulus classifier is configured to classify a stimulus corresponding to a largest canonical correlation analysis (CCA) coefficient among CCA coefficients used in the analysis of covariance as the user's intended visual stimulus.

11. The apparatus of claim 1, wherein:
the visual guide unit is configured to dispose the visual guide at a center of the visual stimulus.

12. A visual stimulus detection method, comprising:
receiving a visual stimulus signal extracted through an electroencephalogram (EEG) analysis with respect to a user gazing at the visual stimulus of a particular frequency, by an apparatus for detecting visual stimulus based on a steady-state visual evoked potential (SSVEP);
disposing a visual guide having a particular form on the visual stimulus, by the apparatus for detecting visual stimulus based on SSVEP;
visual stimulus signal processing to classify the visual stimulus based on the received visual stimulus signal and generate a visual feedback, by the apparatus for detecting visual stimulus based on SSVEP; and
reflecting the visual feedback to the visual guide in real time, by the apparatus for detecting visual stimulus based on SSVEP,
wherein the disposing the visual guide comprises varying a shape of the visual guide in real time based on the visual feedback.

13. The visual stimulus detection method of claim 12, wherein the visual guide has the same frequency as the particular frequency of the visual stimulus.

14. The visual stimulus detection method of claim 13, wherein the disposing the visual guide further comprises varying the shape of the visual guide when the visual stimulus flickers according to the particular frequency.

15. The visual stimulus detection method of claim 12, wherein the visual stimulus signal processing comprises:
extracting a feature of the visual stimulus signal and classifying the visual stimulus based on the extracted feature; and
generating the visual feedback including feedback information related to classification of the visual stimulus.

16. The visual stimulus detection method of claim 15, wherein the generating the visual feedback comprises calculating a slope of a regression line for each visual stimulus through an analysis of covariance with respect to a plurality of visual stimuli including the visual stimulus, and calculating a significance probability with respect to a difference between a first slope appearing largest corresponding to the visual stimulus and a second slope corresponding to another visual stimulus other than the visual stimulus as the visual feedback,
wherein the significance probability is inversely proportional to a magnitude of the difference of the slope.

17. The visual stimulus detection method of claim 16, wherein:
the significance probability has a value varying in a range of 0 or more and 1 or less with respect to the visual stimulus; and
the generating the visual feedback further comprises generating a positive first feedback with respect to the classification as the significance probability is closer to 0 and generating a negative second feedback with respect to the classification as the significance probability is closer to 1.

18. The visual stimulus detection method of claim 17, wherein the disposing the visual guide further comprises varying the shape of the visual guide to a first shape corresponding to the first feedback and varying the shape of the visual guide to a second shape corresponding to the second feedback.

19. The visual stimulus detection method of claim 16, wherein:
the generating the visual feedback further comprises setting all significance probabilities with respect to slope differences between other visual stimuli to 1, excluding the significance probability with respect to the difference between the first slope and the second slope; and
a magnitude of the second slope is subsequently large to magnitude of the first slope.

20. The visual stimulus detection method of claim 12, wherein the disposing the visual guide further comprises disposing the visual guide at a center of the visual stimulus.
